(19) United States
(12) Patent Application Publication
(10) Pub. No.: US 2019/0335987 A1
(43) Pub. Date: Nov. 7, 2019

(54) Disposable Bronchoscope and Method of Use

(71) Applicant: Daniel J. Cook, St. Louis, MO (US)

(72) Inventor: Daniel J. Cook, St. Louis, MO (US)

(21) Appl. No.: 16/387,736

(22) Filed: Apr. 18, 2019

Related U.S. Application Data
(60) Provisional application No. 62/665,714, filed on May 2, 2018.

(51) Int. Cl. A61B 1/267; A61B 1/005; A61B 1/015; A61B 1/00; A61B 1/04

(52) U.S. Cl. CPC A61B 1/2676; A61B 1/0052; A61B 1/015; A61B 1/00114; A61B 1/00124; A61B 1/00119; A61B 1/00105; A61B 1/00016; A61B 1/00009; A61B 1/00027; A61B 1/04

(57) ABSTRACT

A bronchoscope has a handle with a suction port and a flexible scope port. A carrier tube extends from the handle and has first and second passageways. The first passageway communicates with the suction port and the second passageway communicates with the flexible scope port. A flexible scope assembly is insertable into the second passageway of the carrier tube. The flexible scope assembly includes a camera wire with a camera on one end and a transmission connector on an opposite end. The flexible scope assembly has a sheath extending around the camera wire that seals the camera wire and the transmission connector and the camera wire and the camera. The camera includes an imaging device and lighting elements. The flexible scope assembly is removably connectable to the handle at the flexible scope port. The flexible scope assembly may be sanitized when removed from the handle.

### RELATED APPLICATION DATA

This application claims the benefit of U.S. provisional application Ser. No. 62/665,714, filed May 2, 2018, the disclosure of which is incorporated by reference herein.

### FIELD OF THE DISCLOSURE

This disclosure is related to a bronchoscope. More in particular, the disclosure is related to a disposable bronchoscope. More in particular, the disclosure is directed to a disposable bronchoscope with a disposable handle and carrier tube and a re-usable, sanitizable, flexible scope assembly comprising a camera and a transmission connector which is removably insertable in the carrier tube and removably connectable with the handle.

### DESCRIPTION OF THE DRAWINGS

FIG. 1 shows an exemplary disposable bronchoscope in accordance with the disclosure.

FIG. 2 shows a flexible scope assembly of bronchoscope of FIG. 1.

FIG. 3A shows an enlarged view of an end of the flexible scope assembly of FIG. 2 and a transmission connector.

FIG. 3B shows an enlarged view of an opposite end of the flexible scope assembly of FIG. 1 and a camera comprising an imaging device and lighting elements.

FIG. 4 shows a carrier tube of the bronchoscope of FIG. 1.

FIG. 5 shows an enlarged cross-sectional view of the carrier tube taken along lines 5-5 of FIG. 4.

FIG. 6 shows an exploded view of the flexible scope assembly disposed in the carrier tube.

FIG. 7 shows a cross-sectional view of a handle of the bronchoscope taken along lines 7-7 of FIG. 10 with additional detail of the carrier tube and the connection of a suction port of the handle with a passageway of the carrier tube.

FIG. 8 shows a top view of the handle and a flexible scope port and a fluid port formed in the handle.

FIGS. 9 and 10 show cross-sectional views of the handle and the connection of passageway of the carrier tube with the suction port, the fluid port, and the flexible scope port formed in the handle.

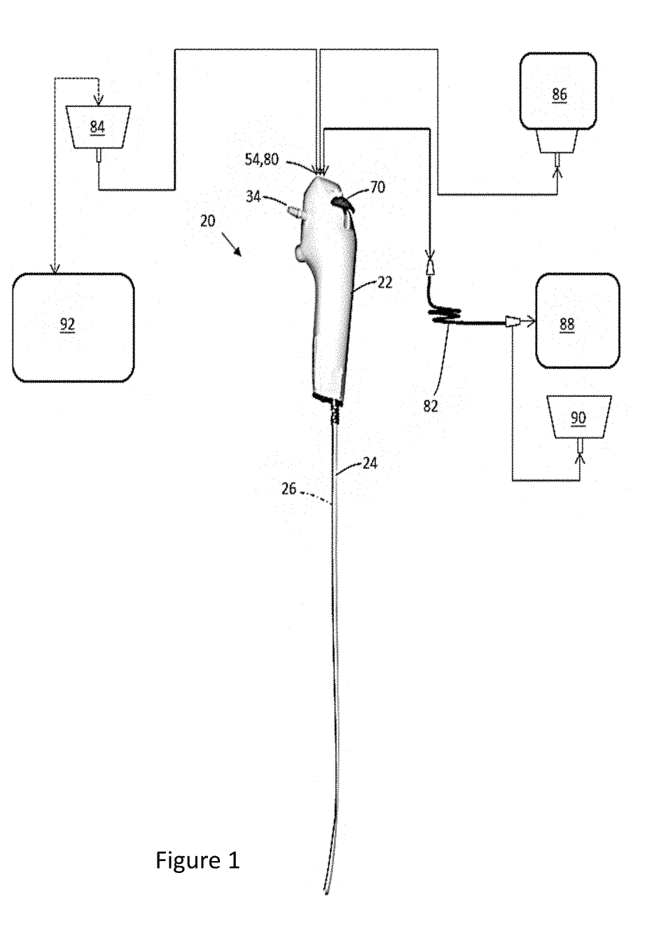

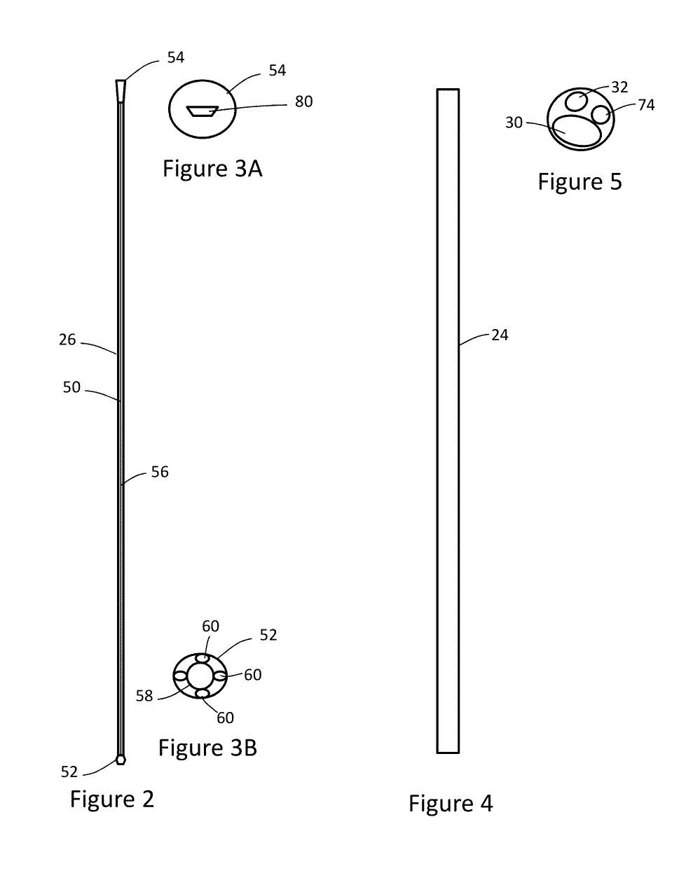

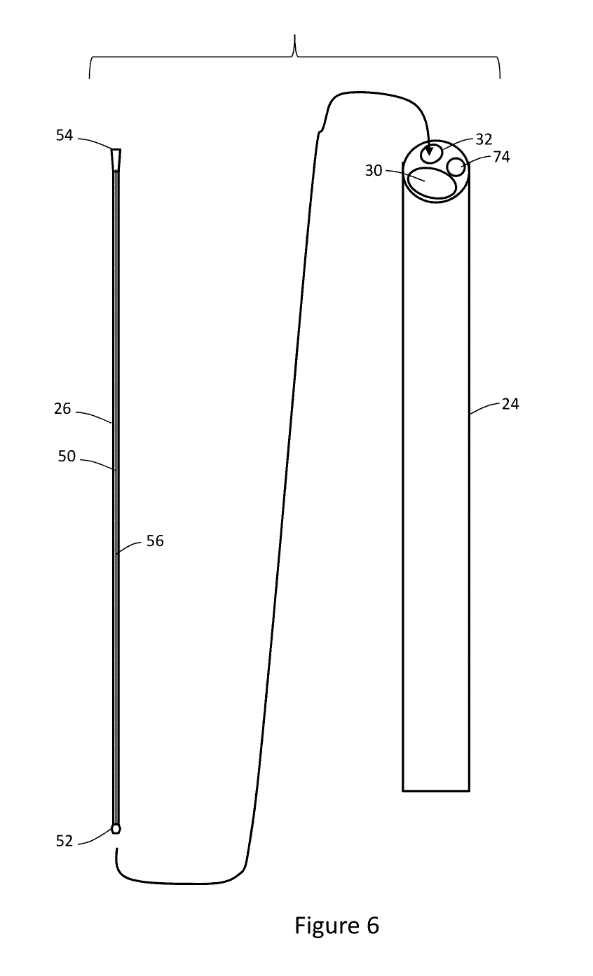

### DETAILED DESCRIPTION

As will become apparent from the description that follows, a bronchoscope 20 is presented that includes a handle 22 and a carrier tube 24, and a flexible scope assembly 26 removably connectable with the handle and removably insertable in the carrier tube. The flexible scope assembly 26 may be removed from the handle 22 and the carrier tube 24. The handle 22 and carrier tube 24 may be discarded after use. The flexible scope assembly 26 may be sanitized and reused with a new handle and carrier tube.

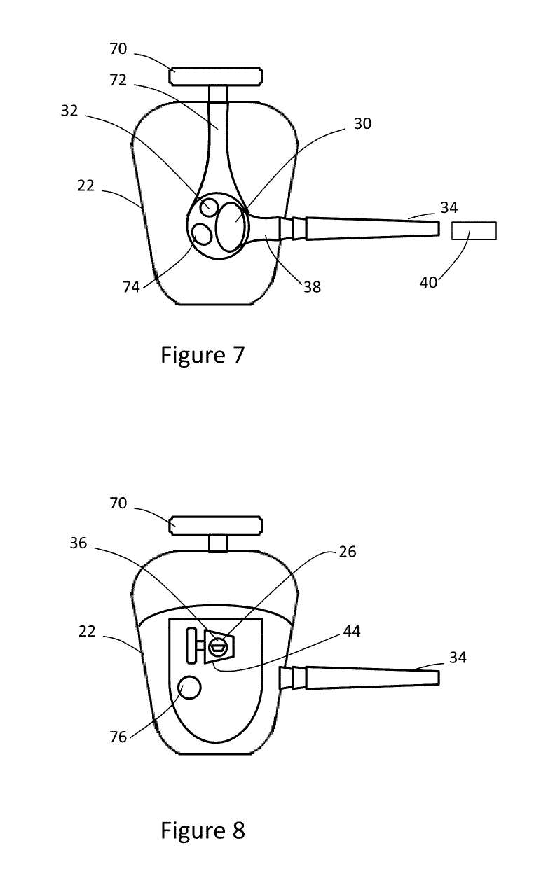

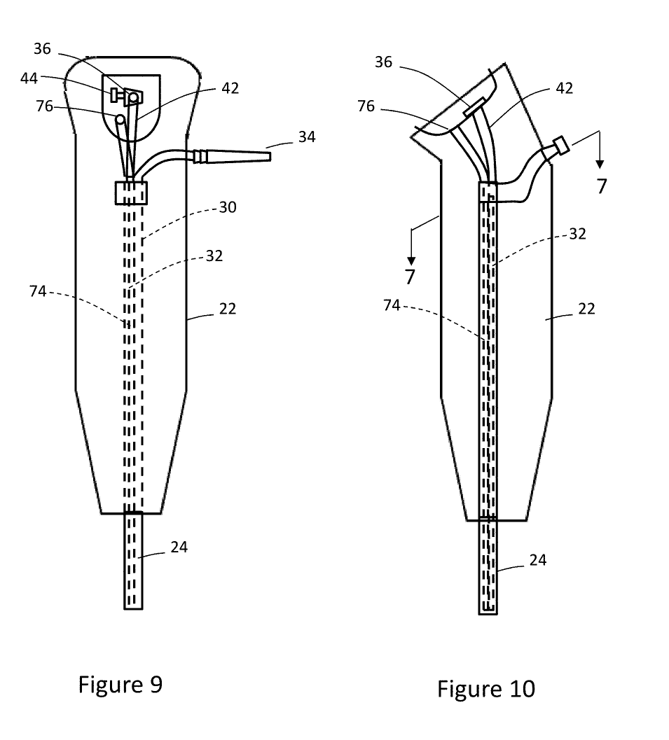

The handle 22 may be integral with the carrier tube 24 or removably connectable to the carrier tube. The carrier tube 24 may extend from the handle 22 and is insertable into the patient. The carrier tube 24 may have first and second passageways 30,32 extending through a length of the carrier tube. The first passageway 30 may communicate with a suction port 34 disposed in the handle 22 when the carrier tube 24 is connected with the handle 22. The second passageway 32 may communicate with a flexible scope port 36 disposed in the handle 22 when the carrier tube 24 is connected with the handle 22. The suction port 34 of the handle 22 may extend from a lateral side of the handle and include conduits 38 which may be aligned in fluid communication with the first passageway 30 when the carrier tube 24 is connected with the handle 22. The conduits 38 may be formed within the monolithic structure of the handle 22 and/or may be separate members disposed within a hollow interior of the handle 22. The suction port 34 may include a barbed style connector to allow installation of a suction tube 40 (FIG. 7) on the suction port 34 on the lateral side of the handle 22.

The flexible scope port 36 may be provided on an exposed part of the handle 22. The flexible scope port 36 communicates with the second passageway 32 of the carrier tube 24. Preferably, the flexible scope port 36 is adjacent the top of the handle to allow easy access for insertion of the flexible scope assembly 26 through the flexible scope port 36 and into the second passageway 32. Also, providing the flexible scope port 36 adjacent the top of the handle 22 allows for the connection of imaging displays and other peripheral devices, and the viewing of imaging displays by professional staff using the bronchoscope, as will be explained. The flexible scope port 36 may comprise conduits 42 formed within the monolithic structure of the handle and/or may comprise separate members disposed within a hollow interior of the handle. The flexible scope assembly 26 may be inserted through an opening of the flexible scope port 36 at the top end of the handle 22. The flexible scope assembly 26 may then be directed through the conduit 42 to the second passageway 32 and extended through the carrier tube 24. The flexible scope assembly 26 may be releasably connected to the handle 22 at the flexible scope port 36. For instance, as shown in the drawings (FIG. 8), the flexible scope port 36 may comprise a releasable connector 44 that engages the flexible scope assembly 26 to releasably connect the flexible scope assembly to the handle at the flexible scope port. The opposite configuration may also be provided where the flexible scope assembly may comprise a releasable connector that engages the flexible scope port to releasably connect the flexible scope assembly to the handle at the flexible scope port. A combination of these elements may also be provided. In one embodiment, the flexible scope port may comprise a push button 46 and spring loaded releasable connector 44 which may engage the flexible scope assembly 26 to releasably connect the flexible scope assembly to the handle 22 at the flexible scope port 36.

The flexible scope assembly 26 may comprise a camera wire 50 having a length with a camera 52 on one end of the length and a transmission connector 54 on an opposite end of the length. The flexible scope assembly 26 may include a sheath 56 extending around the camera wire 50 and sealing the camera wire and the transmission connector 54 at the one end, and sealing the camera wire and the camera 52 at the opposite end. The camera 52 may comprise an imaging device 58 and lighting elements 60 (for instance LEDs) as is known. The camera 52 may comprise a solid state image sensor such as a charge coupled device (“CCD”), a charge injection device (“CID”), or a metal-oxide semiconductor (“MOS”) device (e.g., a complementary metal-oxide semiconductor “CMOS” device). The camera 52 may be controlled (e.g., by a controller and/or software) to adjust image quality (e.g., color hues, white balance, saturation, etc.). The camera 52 may include a lens for desired optical properties. The lighting elements 60 may be presented as an illumination bundle of fibers, and/or by solid-state lighting such as LEDs. In one example, LED lighting may be used to supplement traditional fiber-based illumination, or completely replace it such that no fiber optics are necessary. As such, the camera wire and illumination bundle may comprise only wiring for power and signal transmission with no fiber optics. The flexible scope assembly 26 may be configured for sanitation when removed from the handle. Accordingly, after use of the bronchoscope, the flexible scope assembly 26 may be removed from the carrier tube 24 by releasing the releasable connector 44 connecting the flexible scope assembly 26 to the handle 22 at the flexible scope port 36. The flexible scope assembly 26 may be withdrawn from the carrier tube 24 by pulling the flexible scope assembly on the end with the transmission connector 34 such that the camera 52 passes through the length of the carrier tube and through the conduit 42 and out the flexible scope port 36. The flexible scope assembly 26 may then be sanitized in conventional operating room sanitizing equipment. The handle 22 and carrier tube 24 may be discarded. The flexible scope assembly 26 may be reused with a new handle and carrier tube when a new use is needed.

The handle 22 may include a directional control actuator 70 extending from the handle. The directional control actuator 70 of the handle may be operatively connected (for instance, via a linkage 72) with the carrier tube 24 and configured to move the carrier tube relative to the handle 22. For instance, the directional control actuator 70 may allow for the distal end of the carrier tube to move 130° off centerline in 360 degrees of direction about a center axis extending along a length of the carrier tube. The directional control actuator 70 allows for imaging of a patient when the bronchoscope is in use. The directional control actuator 70 also allows for suction and irrigation as needed via the first passageway 30 and its connection to the suction port 34, and a third passageway 74, if provided in the carrier tube, and its associated conduit 76.

The transmission connector 54 of the flexible scope assembly 26 may include an electronics port 80 for connection of a cable 82 (for instance, a mini HDMI cable), a wireless transmission device 84 and/or an image display device 86. The transmission connector 54 and/or electronics port may be presented in the form of an HDMI connector (e.g., HDMI 2.0), a DVI connector, or an SDI connector (e.g., 12G SDI) or the like, thereby allowing for the images captured by the camera 52 to be displayed on display equipment. The image display device 86 may be coupled directly to the handle 22 at the transmission connector 54. In the alternative, the cable 82 may be inserted into the electronics port 80 and may be connected to an image display device 88 remote to the handle or a wireless transmission device 90 remote to the handle 22.

In the alternative, the wireless transmission device 84 may be removably connected to the electronics port 80 formed on the transmission connector 54 on the handle. The wireless transmission device 84 may be integral with the transmission connector 54 and/or electronics port 80. The wireless transmission device 84,90 may be configured to transmit images from the imaging device 58 of the camera 52 to an image display device 92 remote from the handle. The wireless transmission device 84,90 may include a wireless transmitter and antenna for transmitting video and/or control signals to external equipment. Such wireless communications can include WiFi (e.g., IEEE 802.11a/b/d/g/n/ac), Bluetooth, NFC, ultra wide band (UWB) technology, wireless HDMI, Zigbee, Z-Wave and/or other standard communications protocols. The image display device 92 may include a wireless receiver and antenna configured to receive and decode/process the transmitted signals, to thereby display the video acquired by the camera on the display of the image display device 92. The wireless transmissions of the wireless transmission device 84,90 may alternatively be based on a non-standard or proprietary wireless communications protocol. Computer application software may be provided to allow configuration of the wireless transmission device 84,90 in order to transmit images from the imaging device 58 to an imaging display 92 remote from the handle.

The image display device 88 for displaying images including video images from the camera 52 can include a port for receiving a connector of the wire 82. The insertable image display device 86 may include a port that is connectable with the transmission connector 54 and/or electronics port 80. The wireless transmission device 84,90 may include a port that is connectable with the transmission connector 54 and/or electronics port 80, and/or may include a wireless receiver for receiving the transmitted data comprising the video signal. A signal processor of the image display device 86,88,92 can be connected to a decoder to decode the received video data. For example, a wireless receiver of the image display device 90 can receive the signals transmitted from the wireless transmission device 84,90 and the decoder can decode the encoded data, to thereby provide a video output to the video display of the image display device 92 for viewing. The video display may include a display monitor which may be presented as a standalone unit such as a separate display. And as mentioned above, the video display may include a display monitor which may be presented as an insertable video display device 86. The images from the camera 52 can be transmitted to the image display device 86,88,92 and viewed in real-time and/or recorded by a recording device onto a recording medium (e.g., digital recording medium, such as an SD card) (not shown). The derived video signal can be transmitted in any suitable analog or digital format (e.g., MPEG, AVC, HEVC, etc.) and resolution, framerate, bit depth, color depth, etc. (e.g., 1080p HD at 30fps, 4K UHD 60 at fps, etc.) so long as it can be received and broadcast on the video display for viewing by the user (e.g., surgical team). Likewise, any suitable image standard may be used for captured images, such as JPEG, PNG, and the like.

The wireless transmission device 84,90 and/or the image display device 86,88 may have an integrated power supply to supply power to the camera 52 of the flexible scope 26. In the alternative, a power supply may be connected to the transmission connector 54. The transmission connector 54 may be configured to supply power to the camera 52, including the imaging device 58 and lighting elements 60. The power may be supplied via the wireless transmission device 84,90 or the image display device 86 coupled with the transmission connector 54. Power for certain of the various components (e.g., the wireless transmission devices 84,90, image display devices 86,88,92) can be provided by any configuration of hardwired power (e.g., main power), internal/external batteries, other dedicated power unit, or even inductive coupling. The camera 52, the transmission connector 54, the electronics port 80, and the lighting elements 58 may be powered via the wireless devices and/or image displays, which in turn may be powered by hot-swappable rechargeable batteries, with no external hardwired power required.

As is known, various integrated circuits such as signal processors, controllers, encoders, decoders, photosensors, multiplexers, FPGAs, ASICs/SoCs, etc., can be used to process/convert/transmit/receive signals from the camera 52 and transmission connector 54. For example, a signal processor may integrated with the transmission connector 54 and/or electronics port 80, and used to process the signals generated by the camera 52, as well as to provide an output to an encoder for further processing and display with an image display device 86,88 or wireless transmission via the wireless transmission device 84,90. The encoder output itself may be subject to further processing so as to ultimately provide a video signal to the insertable image display device 86, the cable 82 and the image display device 88, or a wireless transmission device 84,90 for transmission to an image display device 92 for viewing.

In view of the foregoing, it will be seen that the several advantages of the invention are achieved and attained. The embodiments were chosen and described in order to best explain the principles of the disclosure and their practical application to thereby enable others skilled in the art to best utilize the principles in various embodiments and with various modifications as are suited to the particular use contemplated. As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A bronchoscope comprising:
a handle with a suction port and a flexible scope port;
a carrier tube extending from the handle, the carrier tube having first and second passageways extending through the carrier tube, the first passageway communicating with the suction port of the handle, the second passageway communicating with the flexible scope port of the handle; and
a flexible scope assembly comprising a camera wire having a length with a camera on one end of the length and a transmission connector on an opposite end of the length, the flexible scope assembly comprising a sheath extending around the camera wire and sealing the camera wire and the transmission connector and the camera wire and the camera, the camera comprising an imaging device and lighting elements, the flexible scope assembly being removably insertable in the second passageway, the flexible scope assembly being removably connectable to the handle at the flexible scope port, the flexible scope assembly being configured for sanitization when removed from the handle.

2. The bronchoscope of claim 1 wherein the carrier tube has a third passageway communicating with a further fluid port of the handle.

3. The bronchoscope of claim 1 further comprising a directional control actuator extending from the handle, the directional control handle being operatively connected with the carrier tube and configured to move the carrier tube relative to the handle.

4. The bronchoscope of claim 1 wherein the transmission connector of the flexible scope assembly is configured to receive a cable.

5. The bronchoscope of claim 1 wherein the transmission connector of the flexible scope assembly is configured to receive an image display device.

6. The bronchoscope of claim 1 wherein the transmission connector of the flexible scope assembly is configured to receive a wireless transmission device.

7. The bronchoscope of claim 6 wherein the wireless transmission device is configured to transmit images from the imaging device of the camera of the flexible scope assembly to an image display device remote from the handle.

8. The bronchoscope of claim 1 wherein the transmission connector of the flexible scope assembly is configured to supply power to the camera.

9. The bronchoscope of claim 1 wherein the flexible scope port comprises a releasable connector configured to removably connect the flexible scope assembly to the handle.

10. A method comprising:
providing a bronchoscope having a handle and a carrier tube extending from the handle, wherein the carrier tube has first and second passageways extending through the carrier tube, the first passageway communicating with a suction port of the handle, the second passageway communicating with a flexible scope port of the handle;
accessing a flexible scope assembly wherein the flexible scope assembly comprises a camera wire having a length with a camera on one end of the length and a transmission connector on an opposite end of the length, the flexible scope assembly comprises a sheath extending around the camera wire and sealing the camera wire and the transmission connector and the camera wire and the camera, and the camera comprises an imaging device and lighting elements;
inserting the flexible scope assembly in the second passageway; and
removably connecting the flexible scope assembly to the handle at the flexible scope port.

11. The method of claim 10 further comprising removing the flexible scope assembly from the handle after using the bronchoscope.

12. The method of claim 11 further comprising sanitizing the flexible scope assembly after the flexible scope assembly is removed from the handle.

13. The method of claim 12 further comprising disposing the handle and carrier tube after the flexible scope assembly is removed from the handle.

14. The method of claim 13 further comprising:
inserting a sanitized flexible scope assembly into a second carrier tube extending from a second handle, wherein the second carrier tube has first and second passageways extending through the second carrier tube, the first passageway communicates with a suction port of the second handle, the second passageway communicates with a flexible scope port of the second handle; and
removably connecting the sanitized flexible scope assembly to the second handle at the flexible scope port of the second handle.

15. The method of claim 10 further comprising connecting a suction tube to the suction port of the handle in a manner to be in fluid communication with the first passageway of the carrier tube.

16. The method of claim 10 wherein the carrier tube has a third passageway communicating with a second fluid port of the handle; and the method further comprises connecting a fluid source to the second fluid port.

17. The method of claim 10 wherein the bronchoscope comprises a directional control actuator extending from the handle, the directional control actuator being operatively connected with the carrier tube and configured to move the carrier tube relative to the handle; and the method further comprises operating the direction control actuator to move the carrier tube.

18. The method of claim 10 further comprising connecting a cable to the transmission connector of the flexible scope assembly.

19. The method of claim 10 further comprising connecting an image display device to the transmission connector of the flexible scope assembly.

20. The method of claim 10 further comprising connecting a wireless transmission device to the transmission connector of the flexible scope assembly.

21. The method of claim 20 further comprising configuring the wireless transmission device to transmit images from the imaging device of the camera of the flexible scope assembly to an image display device remote from the handle.
